(19) United States
(12) Patent Application Publication
(10) Pub. No.: US 2005/0119632 A1
(43) Pub. Date: Jun. 2, 2005

(54) Absorbent structure featuring high density and flexibility

(76) Inventors: Alisa K. Sierra, Appleton, WI (US); Davis Dang H. Nhan, Appleton, WI (US); Michael J. Niemeyer, Appleton, WI (US); Lawrence H. Sawyer, Neenah, WI (US); Robert A. Stevens, Menasha, WI (US)

Correspondence Address: KIMBERLY-CLARK WORLDWIDE, INC., 401 NORTH LAKE STREET, NEENAH, WI, 54956, US

(21) Appl. No.: 10/980,428

(22) Filed: Nov. 2, 2004

Related U.S. Application Data
(60) Provisional application No. 60/517,169, filed on Nov. 3, 2003.

(51) Int. Cl. A61F 13/15; A61F 13/20

(52) U.S. Cl. 604/367

(57) ABSTRACT

Absorbent structures 50 suitable for incorporation into a variety of disposable absorbent articles are disclosed. The absorbent structures 50 feature a relatively high concentration of superabsorbent material, a relatively high density and a relatively high level of flexibility.

This application claims priority to provisional application Ser. No. 60/517,169 entitled Absorbent Structure Featuring High Density And Flexibility and filed in the U.S. Patent and Trademark Office on Nov. 3, 2003. The entirety of provisional application Ser. No. 60/517,169 is hereby incorporated by reference.

### FIELD OF THE INVENTION

The present invention relates to flexible absorbent structures. More particularly, the present invention relates to flexible absorbent structures having a relatively high density, and relatively high concentrations of superabsorbent material. The absorbent structures may be suited for incorporation into a variety of disposable absorbent articles.

### BACKGROUND OF THE INVENTION

Disposable absorbent articles such as training pants, diapers, adult incontinent garments and the like are well known. As can be appreciated, the absorbent structure is a fundamental component of such articles, and can significantly impact the performance of the article as well as the fit of the article upon the wearer. As such, the improvement of absorbent structures suitable for use in such articles is the subject of a considerable amount of research and development.

For example, a highly desired characteristic of such disposable absorbent articles is thinness. Thinner products are less bulky to wear, fit better under clothing, and are more discreet. Further, thinner products are also more compact in the package, making the products easier for the consumer to carry and store. Compactness in packaging also results in reduced distribution costs for the manufacturer and distributor, including less shelf space required in the store per product unit.

One approach to providing thinner disposable absorbent articles has been to increase the concentration of superabsorbent material present in an absorbent structure. In particular, superabsorbent material may account for approximately half of the absorbent materials contained within an absorbent article. Another conventional approach to providing thinner disposable absorbent articles has been to highly compress the absorbent structure after the formation of the structure. Further, a combination of the above described approaches has also been employed.

Unfortunately, such efforts have not always been completely satisfactory. For example, in some instances such conventional approaches may result in absorbent structures that are relatively stiff and/or inflexible. Absorbent structures that are relatively stiff and/or inflexible are usually considered as not being well-suited for incorporation into disposable absorbent articles such as children's training pants. Consequently, there has remained a need to provide absorbent structures that are relatively flexible and have a relatively high concentration of superabsorbent material. Moreover, it is desired to provide such absorbent structures while maintaining or improving the performance of the structures.

### SUMMARY OF THE INVENTION

In response to the foregoing need, a new absorbent structure has been discovered. For example, in one aspect the present invention is directed to an absorbent structure including at least 60 weight % superabsorbent material. In addition, the absorbent structure defines a density of at least 0.30 g/cm³ and a flexibility of less than 150 grams as determined by the Material Flexibility Test set forth herein.

In another aspect the present invention is directed to an absorbent structure including at least 60 weight % superabsorbent material. In addition, the absorbent structure defines a density of at least 0.30 g/cm³ and an edgewise compression of less than 800 g as determined by the Edgewise Compression Test set forth herein.

In yet another aspect, the present invention is directed to an absorbent structure including at least 60 weight % superabsorbent material. In addition, the absorbent structure defines a flexibility of less than 150 grams as determined by the Material Flexibility Test set forth herein and an edgewise compression of less than 800 g as determined by the Edgewise Compression Test set forth herein.

In still yet another aspect, the present invention is directed to a disposable absorbent article defining a first waist region and a second waist region. The article includes a liquid impermeable outer cover, a fluid permeable liner, and an absorbent structure disposed between the outer cover and the liner. The absorbent structure includes at least 60% superabsorbent material and defines a density of at least 0.30 g/cm³ and a level of flexibility of less than 150 g as determined by the Material Flexibility Test set forth herein. The absorbent article further includes a fastening system configured to join the first waist region to the second waist region to define a waist opening and a pair of leg openings.

The above-mentioned and other aspects of the present invention will become more apparent, and the invention itself will be better understood by reference to the drawings and the following description of the drawings.

### BRIEF DESCRIPTION OF THE DRAWINGS

FIG. 1 representatively illustrates a version of an absorbent structure of the present invention suitable for incorporation into a disposable absorbent article such as children's training pants;

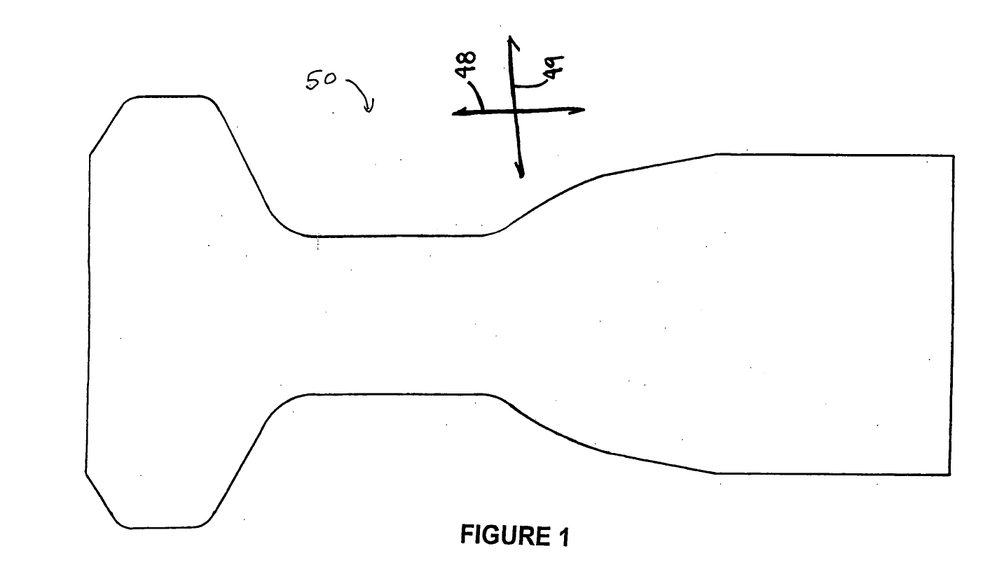

FIG. 2 representatively illustrates a side view of a single-layered absorbent structure similar to that illustrated in FIG. 1;

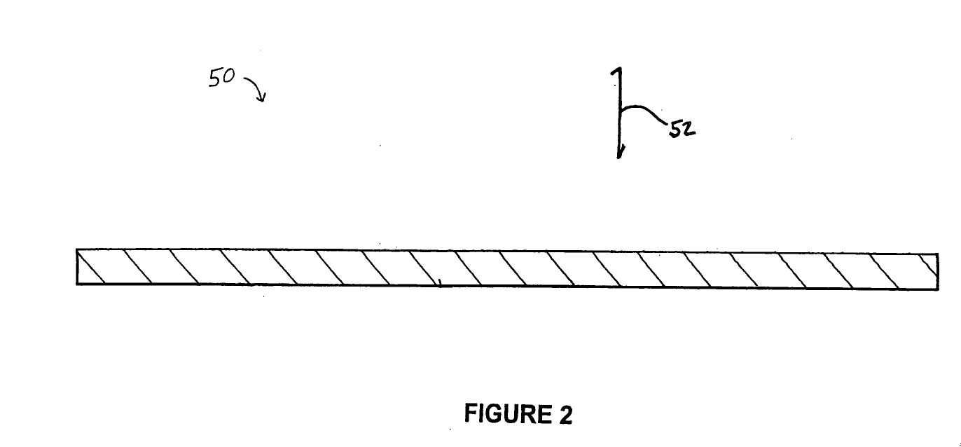

FIG. 3 representatively illustrates a side view of a multi-layered absorbent structure similar to that illustrated in FIG. 1;

FIG. 4 representatively illustrates a side perspective of an absorbent article in the form of a pair of training pants that may include the absorbent structure of the present invention and having a mechanical fastening system fastened on one side of the training pants and unfastened on the opposite side thereof;

FIG. 5 representatively illustrates a bottom plan view of the training pants of FIG. 4 with the pants in an unfastened, unfolded and laid flat condition, and showing the surface of the training pants that faces toward the wearer;

FIG. 6A-6B representatively illustrates a top view and a side view, respectively, of the bottom base board employed for the Fluid Intake and Flowback Evaluation testing;

FIG. 7A-7B representatively illustrates a top view and a side view, respectively, of the top board employed for the Fluid Intake and Flowback Evaluation testing;

FIG. 8A-8B representatively illustrates a top view and a side view, respectively, of a funnel employed for the Fluid Intake and Flowback Evaluation testing;

FIG. 9A-9C representatively illustrates a top view, side view and a perspective view, respectively, of the bottom board, top board and funnel generally configured for use in the Fluid Intake and Flowback Evaluation testing; and

FIG. 10A-10C representatively illustrates a top view, side view and a rear view, respectively of a Saturated Capacity tester.

Corresponding reference characters indicate corresponding parts throughout the several views of the drawings.

### DETAILED DESCRIPTION OF THE INVENTION

The absorbent structure of the various aspects of the present invention is adapted to contain aqueous body exudates such as urine, menses and loose bowel movements. The absorbent can be any structure or combination of components which are generally compressible, conformable, and capable of absorbing and retaining bodily exudates. Accordingly, the absorbent structure of the present invention may be suitable for incorporation into a variety of disposable absorbent articles, such as children's training pants. In particular, the present invention is configured to provide an absorbent structure having a relatively high concentration (i.e., no less than 60%) of particles of superabsorbent material and a relatively high density while yet exhibiting a high degree of flexibility. Moreover, these characteristics and advantages are realized while maintaining or improving the performance of the absorbent structure.

All percentages, ratios and proportions used herein are by weight unless otherwise specified.

As used herein, “Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

As used herein, “Join” and its derivatives refer to the connecting, adhering, bonding, attaching, sewing together, or the like, of two elements. Two elements will be considered to be joined together when they are connected directly to one another or indirectly to one another, such as when each is directly connected to intermediate elements. “Join” and its derivatives include permanent, releasable, or refastenable joinder. In addition, the joinder can be completed either during the manufacturing process or by the end user.

By “particle,” “particles,” “particulate,” “particulates” and the like, it is meant that a material is generally in the form of discrete units. The particles can include granules, pulverulents, powders or spheres. Thus, the particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for use herein. The use of “particle” or “particulate” may also describe an agglomeration including more than one particle, particulate or the like.

Thus, in one aspect, the absorbent structure 50 of the present invention is a matrix of absorbent material that may include hydrophilic fibers. In the various versions of the present invention, many suitable types of wettable, hydrophilic fibrous material can be used to form many of the various component parts of the absorbent structure 50. Examples of suitable hydrophilic fibers include naturally occurring organic fibers composed of intrinsically wettable material, such as cellulosic fibers. Suitable sources of cellulosic fibers include: wood fibers, such as bleached kraft softwood or hardwood, high-yield wood fibers, and ChemiThermoMechanical Pulp fibers, bagasse fibers, milkweed fluff fibers, wheat straw, kenaf, hemp, pineapple leaf fibers, or peat moss. Other hydrophilic fibers, such as regenerated cellulose and curled chemically stiffened cellulose fibers may also be densified to form an absorbent structure that can expand to a higher loft when wetted. Pulp fibers may also be stiffened by the use of crosslinking agents such as formaldehyde or its derivatives, glutaraldehyde, epichlorohydrin, methylolated compounds such as urea or urea derivatives, anhydrides such as maleic anhydride, non-methylolated urea derivatives, citric acid or other polycarboxylic acids.

One example of a suitable hydrophilic fiber is available from Bowater of Coosa River, Ala., U.S.A. as model designation CR1654 and is a bleached, highly absorbent sulfate wood pulp containing primarily soft wood fibers. Other suitable hydrophilic fibers are available from Weyerhauser of Federal Way, Wash., U.S.A. as model designation ND-416 and NB-480.

Other examples of suitable hydrophilic fibers include synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material that has a suitable hydrophilic moiety and is not readily removable from the fiber, or by sheathing a nonwettable, hydrophobic fiber with a hydrophilic polymer during or after the formation of the fiber.

For the purposes of the present invention, it is contemplated that selected blends of the various types of fibers mentioned above may also be employed. Moreover, the fiber selection may instead, or may additionally, include bi-component or bi-constituent fibers that are hydrophilic or have been treated to be hydrophilic and are used to enhance the integrity and/or softness of the absorbent structure by bonding through heat activation.

As used herein, the term “hydrophilic” is intended to describe fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with such a system, fibers having contact angles less than 90° are designated “wettable”, while fibers having contact angles equal to or greater than 90° are designated “nonwettable”.

The hydrophilic fibers of the absorbent structure 50 may be mixed or otherwise incorporated with high-absorbent material such as particles of superabsorbent material. In certain aspects, for example, the absorbent structure 50 includes a mixture of particles of a superabsorbent material and hydrophilic fibers. In certain other aspects, the absorbent structure 50 may include a mixture of particles of a superabsorbent material, natural fibers and synthetic polymer meltblown fibers (a fibrous coform material including a blend of natural fibers and/or synthetic polymer fibers). In a particular aspect, the absorbent structure 50 of the present invention may generally include a mixture of particles of a superabsorbent material, and cellulosic fibers (e.g., wood pulp fibers).

The superabsorbent material may be substantially homogeneously mixed with the hydrophilic fibers, or may be nonuniformly mixed. For example, the concentrations of the superabsorbent material may be arranged in a non-step-wise gradient through a substantial portion of the thickness (i.e., z-direction 52) of the absorbent structure, with lower concentrations toward the bodyside of the absorbent structure and relatively higher concentrations toward the outerside of the absorbent structure. Suitable z-gradient configurations are described in U.S. Pat. No. 4,699,823 to Kellenberger et al. (attorney docket number 7,314), the entire disclosure of which is incorporated herein by reference in a manner that is consistent (i.e., not in conflict) herewith. Alternatively, the concentrations of superabsorbent material may be arranged in a non-step-wise gradient, through a substantial portion of the thickness (z-direction) of the absorbent structure 50, with higher concentrations toward the bodyside of the absorbent structure 50 and relatively lower concentrations toward the outerside of the absorbent structure 50. The superabsorbent material may also be arranged in a generally discrete layer within the matrix of hydrophilic fibers. In addition, two or more different types of superabsorbent material may be selectively positioned at different locations within or along the fiber matrix.

A wide variety of materials can be suitably employed as the superabsorbent material of the present invention. It is desired, however, to employ superabsorbent materials in particle form capable of absorbing large quantities of fluids, such as water, and of retaining such absorbed fluids under moderate pressures.

As used herein, “high-absorbent material,” “superabsorbent material,” “superabsorbent materials” and the like are intended to refer to a water-swellable, water-insoluble organic or inorganic material capable, under the most favorable conditions, of absorbing at least about 10 times its weight and, preferably, at least about 15 times its weight in an aqueous solution containing 0.9 weight percent of sodium chloride. Such materials include, but are not limited to, hydrogel-forming polymers which are alkali metal salts of: poly(acrylic acid); poly(methacrylic acid); copolymers of acrylic and methacrylic acid with acrylamide, vinyl alcohol, acrylic esters, vinyl pyrrolidone, vinyl sulfonic acids, vinyl acetate, vinyl morpholinone and vinyl ethers; hydrolyzed acrylonitrile grafted starch; acrylic acid grafted starch; maleic anhydride copolymers with ethylene, isobutylene, styrene, and vinyl ethers; polysaccharides such as carboxymethyl starch, carboxymethyl cellulose, methyl cellulose, and hydroxypropyl cellulose; poly(acrylamides); poly(vinyl pyrrolidone); poly(vinyl morpholinone); poly(vinyl pyridine); and copolymers and mixtures of any of the above and the like. The hydrogel-forming polymers are preferably lightly cross-linked to render them substantially water-insoluble. Cross-linking may be achieved by irradiation or by covalent, ionic, van der Waals attractions, or hydrogen bonding interactions, for example. A desirable superabsorbent material is a lightly cross-linked hydrocolloid. Specifically, a more desirable superabsorbent material is a partially neutralized polyacrylate salt. Processes for preparing synthetic, absorbent gelling polymers are disclosed in U.S. Pat. No. 4,076,663, issued to Masuda et al., and U.S. Pat. No. 4,286,082, issued to Tsubakimoto et al.

Superabsorbent materials employed in the present invention suitably should be able to absorb a liquid under an applied load. For purposes of this application, the ability of a superabsorbent material to absorb a liquid under an applied load and thereby perform work is quantified as the Absorbency Under Load (AUL) value. The AUL value is expressed as the amount (in grams) of an approximately 0.9 weight percent saline (sodium chloride) solution absorbed by about 0.160 grams of superabsorbent material when the superabsorbent material is under a load. Common loads, further described hereinbelow, include those of about 0.29 pound per square inch, 0.57 pound per square inch, and about 0.90 pound per square inch. Superabsorbent materials suitable for use herein desirably are stiff-gelling superabsorbent materials having an AUL value under a load of about 0.29 pound per square inch of at least about 7; alternatively, at least about 9; alternatively, at least about 15; alternatively, at least about 20; alternatively, at least about 24; and, finally, alternatively, at least about 27 g/g. (Although known to those skilled in the art, the gel stiffness or shear modulus of a superabsorbent material is further described in U.S. Pat. No. 5,147,343, issued to Kellenberger, and European Patent Office Publication No. 0339461, published Nov. 2, 1989, the disclosure of each of which is incorporated herein by reference to the extent that each is consistent (i.e., does not conflict) herewith.) AUL values may be determined by a number of methods known to those of skill in the art. One such method is described in U.S. Pat. No. 5,601,542, issued to Melius et al. (attorney docket number 10,838.2). AUL is believed to be a function of the following factors: (1) gel stiffness while swelling, (2) ability to imbibe the fluid by osmotic and internal electrostatic repulsion forces, (3) surface wettability of the superabsorbent material and (4) particle size distribution when wet.

Superabsorbent materials are typically available from various commercial vendors, such as, for example, Dow Chemical Company or Stockhausen, Inc. In particular, FAVOR SXM 9394 superabsorbent material, available from Stockhausen, Inc. or XUS-40703.02 superabsorbent material, available from Dow Chemical Company, are examples of a suitable superabsorbent for use with the present invention.

Suitably, the superabsorbent material is in the form of particles which, in the unswollen state, have maximum cross-sectional diameters ranging between about 50 and about 1,000 microns; desirably, between about 100 and about 800 microns; more desirably, between about 200 and about 650 microns; and most desirably, between about 300 and about 600 microns, as determined by sieve analysis according to American Society for Testing Materials Test Method D-1921. It is understood that the particles of superabsorbent material may include solid particles, porous particles, or may be agglomerated particles including many smaller particles agglomerated into particles falling within the described size range.

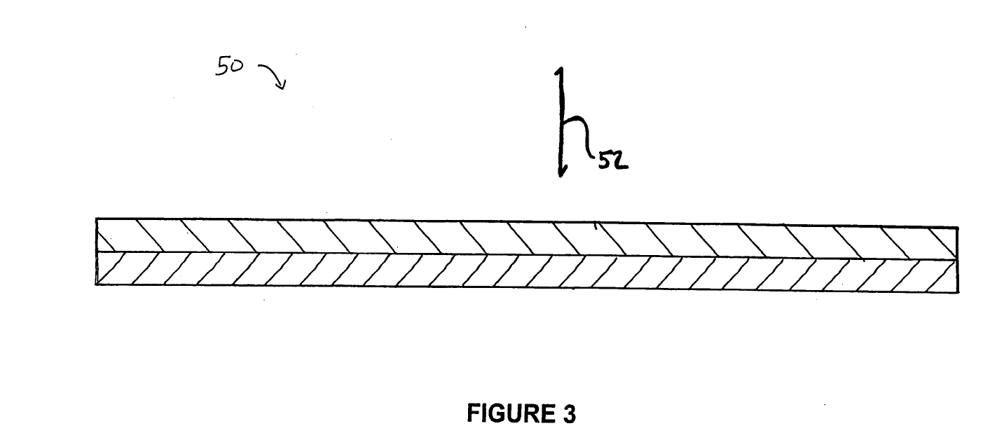

The absorbent structure 50 may be of substantially any shape and size suitable for its intended purpose. Further, as representatively illustrated in FIG. 3, the absorbent structure 50 may also comprise two or more layers, which may be positioned in side-by-side relationship or surface-to-surface relationship, and all or a portion of adjacent webs or layers may be secured together to form the absorbent structure 50.

Further, those of skill in the art will readily appreciate that other suitable arrangements for the absorbent structure 50 of the present invention include configurations having one or more layers (FIG. 3). Each layer may include particles of superabsorbent material, non-superabsorbent material and/or combinations thereof. The layers of any such multi-layered absorbent structure 50 may be connected or otherwise associated together in an operable manner. As used herein when describing layers of an absorbent structure 50, the term “associated” is intended to encompass configurations in which two or more layers are directly in liquid communication with each other, as well as configurations where two or more layers are indirectly in liquid communication with each other by affixing portions of a layer to intermediate members or elements which in turn are affixed to at least portions of another layer. As will be appreciated by one of ordinary skill in the art, one or more layers of an absorbent structure 50 may include other materials such as, for example, woven or nonwoven sheets, scrim netting or other stabilizing structures, superabsorbent material, binder materials, surfactants, selected hydrophobic materials, pigments, lotions, odor control agents or the like, as well as combinations thereof.

The absorbent structure 50 of the present invention may be formed in any conventional manner, such as by being air-formed, air-laid, co-formed, bonded-carded or formed by other known techniques in which fibers and superabsorbent material are commingled to form a non-woven web. For example, the absorbent structure 50 can be a laminate wherein the superabsorbent material is placed in a uniform or patterned array on at least one layer of permeable and hydrophilic fibers or web or between such layers.

The absorbent structure 50 may or may not be wrapped or otherwise encompassed by a suitable tissue or web wrap for maintaining the integrity and/or shape of the absorbent structure 50.

In the various aspects of the absorbent structure 50 of the present invention, the superabsorbent material may provide a certain percentage of the total absorbent structure, by weight. For example, at least a portion of the absorbent structure 50, and suitably substantially the entire absorbent structure 50, may be at least 60% superabsorbent material. Alternatively, at least a portion of the absorbent structure 50, and suitably substantially the entire absorbent structure 50, may be at least 63% superabsorbent material. In still another alternative, at least a portion of the absorbent structure 50, and suitably substantially the entire absorbent structure 50, may be at least 65% superabsorbent material. In still yet another alternative, at least a portion of the absorbent structure 50, and suitably substantially the entire absorbent structure 50, may be at least 70% suberabsorbent material. In addition, at least a portion of the absorbent structure 50, and suitably substantially the entire absorbent structure 50, may be from 60-90% superabsorbent material; in particular from 60-75% superabsorbent material; more particularly 62-70% superabsorbent material; and still more particularly between 62-68% superabsorbent material. By contrast, many conventional absorbent structures, for example absorbent structures found in children's training pants, may be approximately 40-55% superabsorbent material. The percentage of superabsorbent material found absorbent structures of the present invention may be accomplished by increasing the amount of superabsorbent material included in the absorbent structure (i.e., increasing the superabsorbent material add-on) and/or decreasing the amount of the other components that may make up the absorbent structure 50. The relatively high levels of superabsorbent material included in the absorbent structure 50 of the present invention provide an operative level of absorbency while yet allowing the absorbent body to remain thin and less bulky.

Further, the absorbent structure 50 of the present invention may define a density of no less than 0.30; alternatively, no less than 0.33; alternatively, no less than 0.35; alternatively no less than 0.37, alternatively, no less than 0.40; alternatively, no less than 0.42 or finally, alternatively, no less than 0.45 g/cm³. In addition, the absorbent structure 50 may define a density of between 0.30 and 0.45 g/cm³; in particular, a density of between 0.30 and 0.43 g/cm³; more particularly 0.33 and 0.43 g/cm³; still more particularly 0.35 and 0.43 g/cm³; and still yet more particularly between 0.35 and 0.40 g/cm³. By contrast, many conventional absorbent structures, for example absorbent structures found in children's training pants, generally have a density of between 0.30 and 0.35 g/cm³. Moreover, it can be readily appreciated that the elevated densities described herein can be combined with an elevated percentage of superabsorbent material as described above.

Without being bound to any particular theory, the high percentage of superabsorbent found in the absorbent structure 50 of the present invention combined with an elevated density yields an absorbent structure that is thin and flexible, and thus provides improved fit and comfort to the wearer, along with improved discretion. In addition, the high percentage of superabsorbent in the structure along with the elevated density surprisingly provided an absorbent body with superior dry pad integrity. Moreover, these results were obtained where absorbent bodies having a high density but lower levels of superabsorbent were generally found to be stiff and non-compliant, while absorbent bodies having a high superabsorbent content but lower density would often present a shifting, grainy surface texture to the wearer resulting from poor pad integrity.

Therefore, the present invention may provide a flexible and compliant absorbent structure 50 as indicated by the Material Flexibility Test set forth herein. It should be noted that according to the Material Flexibility Test, the level of flexibility is inversely proportionate to the amount of force required to deform the test specimen. As such, at least a region of the absorbent structure 50 may have a level of flexibility of less than 250 g as measured by the Material Flexibility Test described herein. In particular, at least a region of the absorbent structure 50 may have a level of flexibility of less than 200 g; more particularly, of less than 150 g; still more particularly, of less than 100 g; and finally, of less than 50 g as measured by the Material Flexibility Test described herein.

In addition, the absorbent structure 50 of the present invention may define a caliper in the Z direction 52 (i.e. the direction perpendicular to the plane created by the longitudinal and lateral directions 48, 49). Specifically, the absorbent structure 50 may define a caliper in the Z direction 52 of between 1.0 and 2.5 mm as measured by the Caliper Test described herein. Alternatively, the absorbent structure 50 may define a caliper in the Z direction 52 of between 1.5 and 2.0 mm as measured by the Caliper Test described herein; or in yet another alternative the absorbent structure 50 may define a caliper in the Z direction of between 1.7 and 1.9 mm as measured by the Caliper Test described herein.

Surprisingly, and as mentioned above, the absorbent structure 50 of the present invention may further provide a high level of integrity despite the elevated levels of superabsorbent material found in the structure. For example, absorbent bodies having elevated levels of superabsorbent material often can exhibit superabsorbent material shakeout, and may further be generally prone to separation and or break-up, particularly when undergoing the stresses associated with an absorbent garment in use. Nonetheless, the absorbent structure 50 of the present invention can feature pad integrity comparable to conventional absorbent structures. For example, this pad integrity is demonstrated by the ability of the absorbent structure 50 of the present invention to be subjected to the Edgewise Compression Test described herein. In particular, the Edgewise Compression Test calls for a sample of the absorbent body to be bent and stapled into a cylindrical configuration. By comparison, an absorbent body having elevated levels of superabsorbent but a lower density value would generally not be capable of being formed into a cylinder for a lack of dry pad integrity.

In addition, as demonstrated by the Edgewise Compression Test, this desirable pad integrity is provided without sacrificing flexibility in the absorbent structure 50. The Edgewise Compression Test described herein measures the peak force level needed to compress the edges of the abovementioned cylinder of absorbent material until the cylinder is 50% of its original height. As such, according to this test, the flexibility of an absorbent body is inversely proportional to the amount of force measured by the test. Thus, the lower the resulting force, the more flexible the absorbent body. Suitably, the absorbent structure 50 of the present invention may define an edgewise compression of less than 1000 g. Alternatively, the absorbent structure 50 may define an edgewise compression of less than 800 g, in another alternative less than 700 g, in yet another alternative less than 600 g, and in still yet another alternative, less than 500 g, and suitably less than 400 g. In addition, absorbent products, such as training pants 20, that include the absorbent structure 50 of the present invention may suitably define an edgewise compression of less than 2000 g, and still more suitably, less than 1400 g.

Moreover, in addition to the above benefits, the absorbent structure 50 of the present invention may provide absorbent capabilities suitable for use in disposable absorbent articles, such as children's training pants, as determined by the Saturated Capacity Test set forth herein. For instance, the absorbent structure 50 may have an overall absorbent capacity as determined by the Saturated Capacity (SAT CAP) test of at least 300 g. Alternatively, the absorbent structure 50 may have an overall absorbent capacity of at least 400 g; alternatively at least 700 g; alternatively at least 1000 g; and finally, alternatively at least 1500 g according to the SAT CAP Test set forth herein. Moreover, the absorbent structure 50 of the present invention may have a basis weight of no less than 400; alternatively, no less than 500; alternatively no less than 600; or finally, alternatively, no less than 700 g/m².

The various aspects, benefits, and versions of the absorbent structure 50 will be described in the context of a disposable absorbent article, such as a disposable children's training pant. It is, however, readily apparent that one or more versions of the present invention could also be employed with other disposable absorbent articles, such as feminine hygiene articles, infant diapers, incontinence garments and the like. Typically, disposable absorbent articles are intended for limited use and are not intended to be laundered or otherwise cleaned for reuse. Disposable training pants, for example, are discarded after becoming soiled by the wearer.

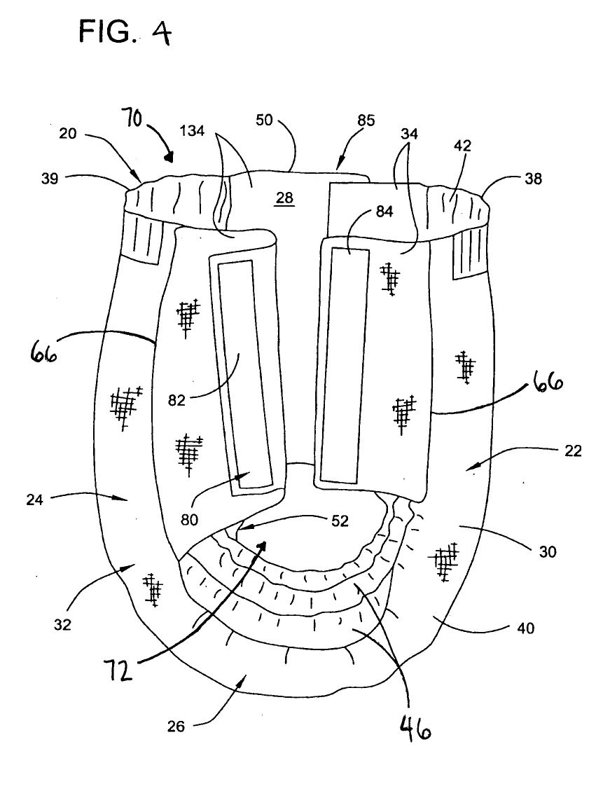

Referring now to the drawings and in particular to FIG. 4, children's toilet training pants including an absorbent structure 50 of the present invention is representatively illustrated and is indicated in its entirety by the reference numeral 20. By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al.; and U.S. Pat. No. 6,645,190 issued Nov. 11, 2003 to Olson et al., which are incorporated herein by reference to the extent they are consistent (i.e., not in conflict) herewith.

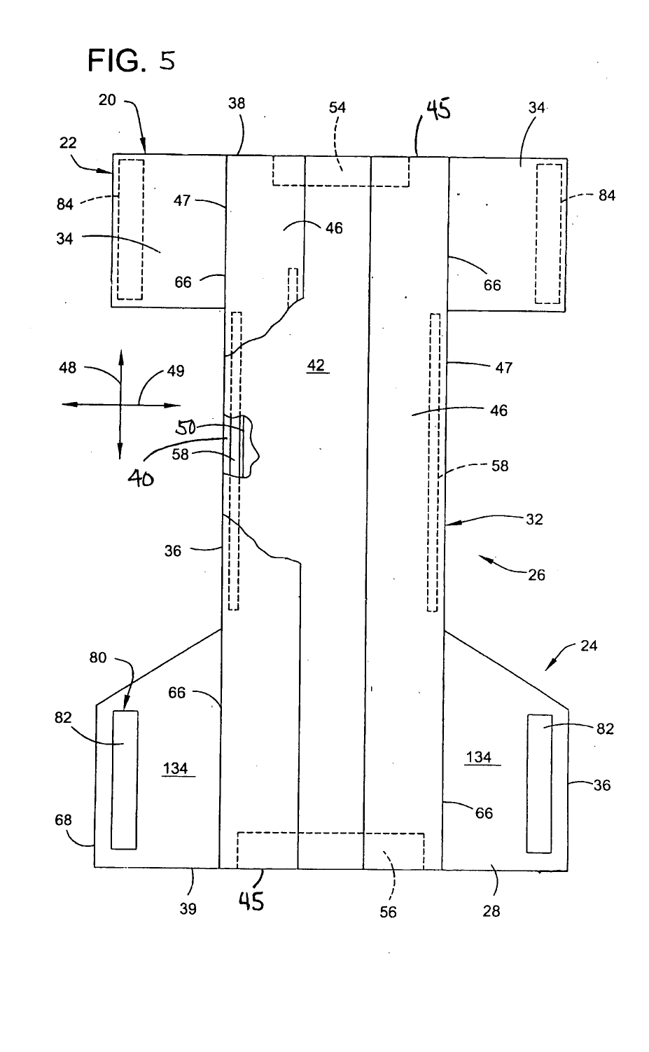

The training pants 20 as illustrated in FIG. 5 to define a longitudinal direction 48 and a lateral direction 49 perpendicular to the longitudinal direction. The pants 20 further define first and second waist regions, otherwise referred to herein as a front waist region 22 and a back waist region 24, and a center region, otherwise referred to herein as a crotch region 26, extending longitudinally between and interconnecting the front and back waist regions 22, 24. The pants 20 also define an inner surface 28 adapted in use (e.g., positioned relative to the other components of the pants 20) to be disposed toward the wearer, and an outer surface 30 opposite the inner surface. The front and back waist regions 22, 24 are those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. With additional reference to FIG. 5, the training pants 20 has a pair of laterally opposite side edges 36 and a pair of longitudinally opposite waist edges, respectively designated front waist edge 38 and back waist edge 39.

The illustrated pants 20 may include a central absorbent assembly, generally indicated at 32, having a pair of laterally opposite front side panels 34 extending laterally outward at the front waist region 22 and a pair of laterally opposite back side panels 134 extending laterally outward at the back waist region 24. The central absorbent assembly 32 is illustrated in FIG. 5 as being generally rectangular. However, it is contemplated that the absorbent assembly 32, may be other than rectangular, such as hourglass shaped, T-shaped, 1-shaped, and the like without departing from the scope of this invention.

The central absorbent assembly 32 of the illustrated aspect has a pair of longitudinally opposed end edges 45, which at least form portions of the front and back waist edges 38, 39, and a pair of laterally opposed side edges 47 which at least form portions of the pants side edges 36. For further reference, arrows 48 and 49 depict the orientation of the longitudinal direction and the transverse or lateral direction, respectively, of the training pants 20.

Still referring to FIGS. 4 and 5, the central absorbent assembly 32 includes an outercover 40 and a bodyside liner 42 that may be joined to the outercover 40 in a superposed relation therewith by adhesives, ultrasonic bonds, thermal bonds or other conventional techniques. The liner 42 can be generally adapted, i.e., positioned relative to the other components of the pants 20, to be disposed toward the wearer's skin during wear of the pants. The absorbent assembly 32 may further include the absorbent structure 50 (FIG. 5) disposed between the outercover 40 and the bodyside liner 42 for absorbing liquid body exudates exuded by the wearer, and may further include a pair of containment flaps 46 (FIG. 5) secured to the bodyside liner 42 for inhibiting the lateral flow of body exudates.

With the training pants 20 in the fastened position as partially illustrated in FIG. 4, the front and back side panels 34, 134 can be connected together by a fastening system 80 that is configured to join the waist regions and define a three-dimensional pants configuration having a waist opening 70 and a pair of leg openings 72. The front and back side panels 34 and 134, upon wearing of the pants 20, thus include the portions of the training pants 20 which are positioned on the hips of the wearer. The waist edges 38 and 39 of the training pants 20 are configured to encircle the waist of the wearer and together define a waist opening 70 of the pants.

The elasticized containment flaps 46 define a partially unattached edge which assumes an upright configuration in at least the crotch region 26 of the training pants 20 to form a seal against the wearer's body. The containment flaps 46 can be located along the absorbent assembly side edges 47, and can extend longitudinally along the entire length of the absorbent assembly 32 or may only extend partially along the length of the absorbent assembly. Suitable constructions and arrangements for the containment flaps 46 are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

To further enhance containment and/or absorption of body exudates, the training pants 20 may also suitably include a front waist elastic member 54, a rear waist elastic member 56, and leg elastic members 58, as are known to those skilled in the art. The waist elastic members 54 and 56 can be operatively joined to the outercover 40 and/or the bodyside liner 42 along the waist edges 38, 39, and can extend over part or all of the waist edges 38, 39. The leg elastic members 58 can be operatively joined to the outercover 40 and/or the bodyside liner 42 along the opposite side edges 36 and positioned in the crotch region 26 of the training pants 20.

The waist elastic members 54, 56 and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA® and available from E. I. Du Pont de Nemours and Company, Wilmington, Del., U.S.A.

In configurations where the side panels 34, 134, are separately attached, the panels can be permanently bonded along seams 66 to the central absorbent assembly 32 in the respective front and back waist regions 22 and 24. More particularly, as representatively illustrated in FIG. 5, the front side panels 34 can be permanently bonded to and extend transversely outward beyond the side edges 47 of the absorbent assembly 32 at the front waist region 22, and the back side panels 134 can be permanently bonded to and extend transversely outward beyond the side margins of the absorbent assembly at the back waist region 24. The side panels 34 and 134 may be bonded to the absorbent assembly 32 using attachment means known to those skilled in the art such as adhesive, thermal or ultrasonic bonding.

Alternatively, the side panels 34 and 134 can be formed as an integral portion of a component of the absorbent assembly 32. For example, the side panels 34, 134 can include a generally wider portion of the outercover 40, the bodyside liner 42, and/or other components of the absorbent assembly 32. In any event, the front and back side panels 34 and 134 can be permanently bonded together to form the three-dimensional configuration of the pants 20, or be releasably connected with one another such as by the fastening system 80 of the illustrated aspects.

In configurations where the side panels 34, 134 are separately attached, the side panels may be provided by an elastic material capable of stretching at least in a direction generally parallel to the lateral direction 49 of the training pants 20. Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference to the extent that they are consistent (i.e., not in conflict) herewith.

The fastening system 80 may include laterally opposite first fastening components 82 adapted for refastenable engagement to corresponding second fastening components 84. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the pants 20 in its three-dimensional configuration.

The fastening components 82, 84 may be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include loop fasteners and the second fastening components 84 include complementary hook fasteners. Alternatively, the first fastening components 82 may include hook fasteners and the second fastening components 84 may be complementary loop fasteners. In another aspect, the fastening components 82, 84 can be interlocking similar surface fasteners, or adhesive and cohesive fastening elements such as an adhesive fastener and an adhesive-receptive landing zone or material; or the like. Although the training pants 20 illustrated in FIG. 4 indicate the back side panels 134 overlapping the front side panels 34 upon connection thereto, the training pants 20 can also be configured so that the front side panels 34 overlap the back side panels 134 when connected. One skilled in the art will recognize that the shape, density and polymer composition of the hooks and loops may be selected to obtain the desired level of engagement between the fastening components 82, 84. Optionally, either one or both of the fastening components 82, 84 may be provided by one of the inner or outer surfaces 28 and 30 of the side panels 34 and 134. When engaged, the fastening components 82, 84 of the illustrated aspect define refastenable engagement seams 85.

The outercover 40 suitably includes a material which is substantially liquid impermeable. The outercover 40 can be a single layer of liquid impermeable material, or alternatively can be a multi-layered laminate structure in which at least one of the layers is liquid impermeable. For instance, the outercover 40 can include a liquid permeable outer layer and a liquid impermeable inner layer that are suitably joined together by a laminate adhesive, ultrasonic bonds, thermal bonds, or the like. Suitable laminate adhesives, which can be applied continuously or intermittently as beads, a spray, parallel swirls, or the like, can be obtained from Bostik Findley Adhesives, Inc., of Wauwatosa, Wis., U.S.A., or from National Starch and Chemical Company, Bridgewater, N.J. U.S.A. The liquid permeable outer layer can be any suitable material and is desirably one that provides a generally cloth-like texture. One example of such a material is a 20 gsm (grams per square meter) spunbond polypropylene nonwoven web. The outer layer may also be made of those materials of which the liquid permeable bodyside liner 42 is made.

The inner layer of the outercover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outercover 40 when a single layer, prevents waste material from wetting articles, such as bed sheets and clothing, as well as the wearer and caregiver. A suitable liquid impermeable film for use as a liquid impermeable inner layer, or a single layer liquid impermeable outercover 40, is a 0.02 millimeter polyethylene film commercially available from Pliant Corporation of Schaumburg, Ill., U.S.A.

The outercover 40 can be suitably sized (e.g., in length and width) larger than the absorbent structure 50 to extend outward beyond the periphery thereof. For example, the outercover 40 may extend outward beyond the absorbent structure 50 periphery a distance in the range of about 1.3 centimeters to about 2.5 centimeters (about 0.5 to 1 inch).

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent structure 50. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The pants 20 may also include an absorbent disposed between the outercover 40 and the bodyside liner 42. The absorbent can be any structure or combination of components which are generally compressible, conformable, non-irritating to a wearer's skin, and capable of absorbing and retaining liquids and certain body wastes. Suitably, the absorbent may be the absorbent structure 50 of the present invention.

In certain aspects, a surge management layer (not shown) may be optionally located adjacent the absorbent structure 50 and attached to various components in the article 20 such as the absorbent structure 50 or the bodyside liner 42 by methods known in the art, such as by adhesive. A surge management layer helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

The absorbent structure 50 of the present invention will now be further described and further advantages will become apparent in view of the “Examples” and “Testing” sections set forth below.

### EXAMPLES

The following Examples describe various versions of the invention. Other versions within the scope of the claims herein will be apparent to those skilled in the art from consideration of the specification or practice of the invention as disclosed herein. It is intended that the specification, together with the Examples, be considered exemplary only, and are not intended to limit the scope and spirit of the invention.

In each of the following examples, the absorbent structures were produced using conventional air-form processing equipment known to those of skill in the art. For example, absorbent materials such as hydrophilic fibers and superabsorbent material may be entrained in an airstream. The fibers and superabsorbent material may then be formed into an absorbent structure upon a screen by drawing the airstream through the screen via an air pressure differential across the screen. Examples of such equipment and processes are described in U.S. Pat. No. 4,666,647 issued May 19, 1987; and 6,330,735 issued Dec. 18, 2001.

### Example 1

The training pants of Example 1 generally corresponded to the structure of commercial PULL-UPS® training pants which was available in May, 2003, except that the absorbent structure 50 was provided by an absorbent structure of the present invention. That is, the absorbent structure of this example contained superabsorbent material in an amount of between 56 and 70 weight %, with particular superabsorbent material percentages being provided in the Table below. The superabsorbent material was SXM-9394 available from Stockhausen, Inc., of Greensboro, N.C. The fiber utilized was ND-416 available from Weyerhauser of Federal Way, Wash. The absorbent structure was contained within a 0.48 osy tissue available from Cellu-Tissue of Neenah, Wis. The densities of the absorbent structures of this example were between 0.33-0.40 g/cm³. Methods of densifying absorbent structures are well known in the art. For example, the absorbent structure may be passed between a set of rolls. The gap between the nip rolls may be adjusted such that the desired density for the absorbent structure is achieved. Further attributes are provided in the Table below.

### Example 2

The training pants structure of Example 2 was substantially the same as the training pants structure of Example 1, except for the nature of the absorbent structure 50. That is, the absorbent structure of this example contained superabsorbent material in an amount of between 55 and 70 weight %, with particular superabsorbent material percentages being provided in the Table below. The superabsorbent material was SXM-9394 available from Stockhausen, Inc., of Greensboro, N.C. The fiber utilized was ND-416 available from Weyerhauser of Federal Way, Wash. The absorbent structure was contained within a 0.48 osy tissue available from Cellu-Tissue of Neenah, Wis. The densities of the absorbent structures of this example were between 0.31-0.40 g/cm³. Further attributes are provided in the Table below.

### Example 3

The training pants structure of Example 3 was substantially the same as the training pants structure of Example 1, except for the nature of the absorbent structure 50. That is, the absorbent structure of this example contained superabsorbent material in an amount of between 55 and 71 weight %, with particular superabsorbent material percentages being provided in the Table below. The superabsorbent material was SXM-9394 available from Stockhausen, Inc., of Greensboro, N.C. The fiber utilized was ND-416 available from Weyerhauser of Federal Way, Wash. The absorbent structure was contained within a 0.48 osy tissue available from Cellu-Tissue of Neenah, Wis. The densities of the absorbent structures of this example were between 0.33-0.38 g/cm³. Further attributes are provided in the Table below.

### Example 4

The training pants structure of Example 4 was substantially the same as the training pants structure of Example 1, except for the nature of the absorbent structure 50. That is, the absorbent structure of this example contained superabsorbent material in an amount of between 52% and 66%, with particular superabsorbent material percentages being provided in the Table below. The superabsorbent material was XUS-40703.02 for the male product codes, available from Dow Chemical Company and SXM-9394 for the female product codes, available from Stockhausen, Inc., of Greensboro, N.C. The fiber utilized was NB-480 available from Weyerhauser of Federal Way, Was. The absorbent structure was contained within a 0.48 osy tissue available from Cellu-Tissue of Neenah, Wis. The densities of the absorbent structures of this example were between 0.33-0.44 g/cm³. Further attributes are provided in the Table below.

### Test Methods

### Fluid Intake and Flowback Evaluation (FIFE)

Equipment & Materials:

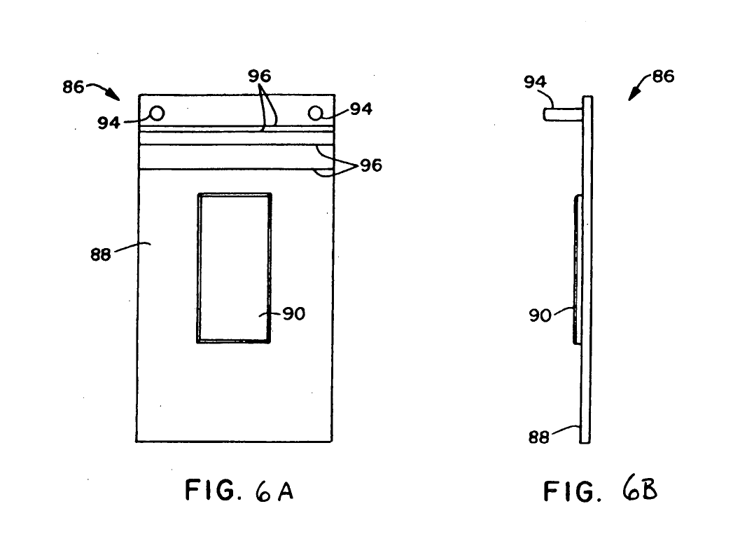

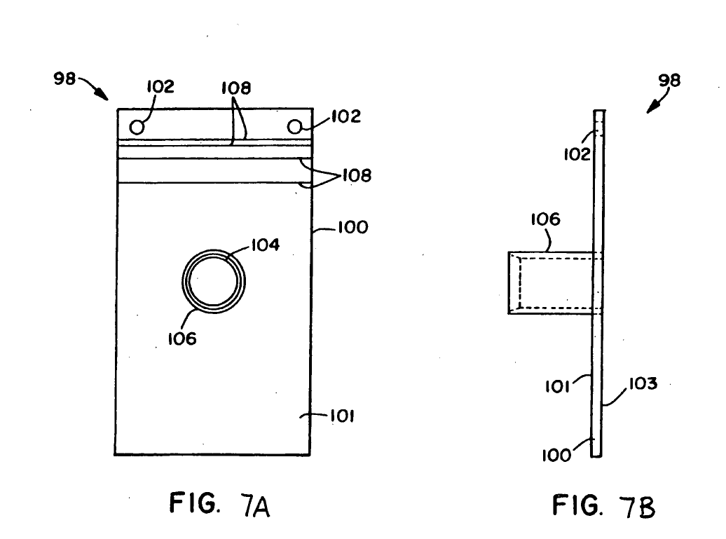

1. FIFE Boards. See FIGS. 6A-6B and 7A-7B: As representatively illustrated in FIGS. 6A and 6B, bottom FIFE board 86 includes a rectangular shaped base member 88 and a smaller, rectangular shaped platform member 90. The base member has an overall length (top to bottom of the figure) of 14 inches, an overall side-to-side width of 8 inches and a thickness of 0.34 inches. Platform member 90 has a length of 6 inches, a width of 4 inches and a thickness of 0.22 inches. The platform member is centered onto the top surface of base 88 and secured in place, such as by adhesive bonding. The four, peripheral top edges of platform 90 are shaped with a 0.06 inch by 45 degree chamfer. Rectangular base 88 includes a pair of 0.5 inch diameter, cylindrical rods 94 which are press fitted into mating holes and secured in place with suitable attachment means, such as adhesive bonding. The center of each rod is positioned 0.75 inch from the top, end edge of the base member, and 0.75 inch from the immediately adjacent side edge of the base member. The rods extend about 1.63 inches above the surface of the base member, and the uppermost exposed edges of rods 94 are rounded with a contour radius of about 0.16 inches. A series of four reference lines 96 are scribed into the top surface of base 88 and extend laterally across the width of the base member. The scribe lines are parallel and spaced from the top, end edge of base 88 by distances of 1.25 inches, 1.50 inches, 2.00 inches and 3 inches, respectively. The components of bottom FIFE board 86 are composed of a suitable water resistant material, such as Lexan plastic.

Top FIFE board 98 includes a top plate 100 and a cylindrical tube 106 which extends generally perpendicular from the plane defined by uppermost, top surface of the top plate. Top plate 100 is generally rectangular in shape and is sized with substantially the same length, width and thickness as bottom FIFE board 86. The top plate includes a pair of 0.53 inch diameter through holes 102 which are located adjacent the top edge of plate 100 and configured to slip over rods 94 in bottom FIFE board 86 to appropriately locate top FIFE board 98 in a substantially congruent, coextensive position over bottom FIFE board 86.

A series of four reference lines 108 are inscribed into a top surface of plate 100 and extend linearly in the transverse direction across the width of the top plate. The scribe lines are parallel and spaced from the top, end edge of base 88 by distances of 1.25 inch, 1.50 inch, 2.00 inch and 3 inch, respectively. The medial section of top plate 100 includes a circular hole which is sized to accept the placement of cylindrical tube 106. Tube 106 has a 2.5 inch outside diameter, a 2.0 inch inside diameter and an overall length of 3.75 inch. The tube is press fitted and attached in place within center hole 104 by suitable attachment means, such as adhesive bonding. Hole 104 is centered with respect to both the length and width of the top plate.

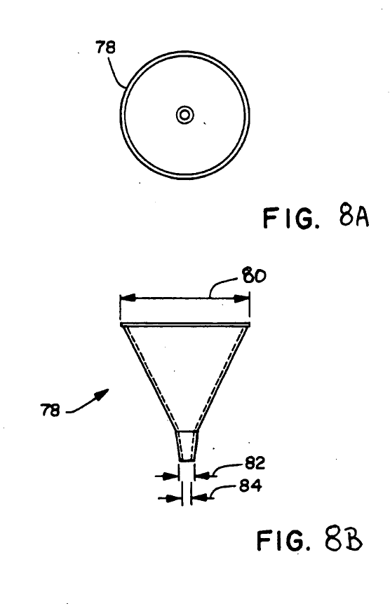

Tube 106 projects generally perpendicular from the top surface 101 of plate 100 and extends through the thickness of the plate to protrude a small distance of about 0.03 inches past bottom surface 103 of plate 100. The upper, entrance edge of tube 106 has an internal chamfer which generally matches the conical shape of the associated funnel representatively illustrated in FIG. 8. Similar to the components of bottom FIFE board 86, the components of top plate 100 are composed of a suitable water resistant material, such as Lexan plastic.

2. Four ounce Funnel; see FIGS. 8A and 8B. Funnel 78 has inlet diameter 79 of 3.25 inches, a funnel throat diameter 82 of 0.438 inches and a spout outlet diameter 84 of 0.25 inches. The given measurements are inside diameters.

3. Blotter papers; two per sample, cut to 8.9 by 30.5 cm rectangles, such as white Verigood grade 300 g/m², available from Georgia Pacific Corp as part number 411-01-12.

4. Specimen: The specimen should be able to lie flat without excessive wrinkles. If elastics are present that prevent the structure from lying flat, these can be clipped at about 1 inch intervals, taking care to avoid clipping into the absorbent region. The specimen should be marked with a permanent marker to show a circle 51 mm in diameter, centered along the longitudinal centerline and at a position set back from the front waist edge of the absorbent by 37% of the total length of the absorbent (for girl products) and 30% of the total length of the absorbent (for boy products).

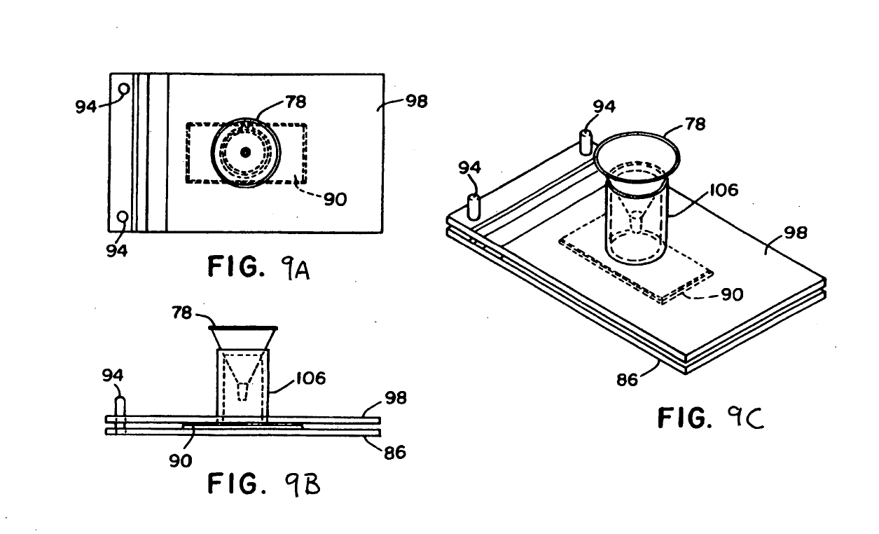

Testing Procedure: (FIGS. 9A-9C)

1. Place the specimen on the bottom FIFE board.

2. Align the specimen so the marked target zone is in the center of the 3×6 inch raised platform.

3. Place the top FIFE board over the target, making sure there are no apparent wrinkles in the liner under the Board, and centering marked target zone under the cylinder. Press lightly on the board to impress the cylinder ridge (on underside of board) into the specimen.

4. Place the funnel into the cylinder. The funnel must be perpendicular to the specimen and in the center of the target zone area. This is determined by sighting through the end of the funnel.

5. Measure the appropriate amount of testing liquid using the dispenser and dispense into the beaker.

6. Pour the liquid from the beaker into the funnel and onto the target area. Avoid pouring directly onto the target area; instead, the liquid should be allowed to contact the side of the funnel. Start the stopwatch when the liquid hits the funnel. NOTE: Pour the liquid as fast as possible without overflowing the funnel.

7. As soon the funnel is empty, remove it.

8. Observe the liquid intake through the cylinder top. Stop the stopwatch the moment no liquid is visible on the specimen surface.

9. Record the time to 0.01 second.

10. Immediately reset the timer for 20 minutes and start it. As the timer runs, prepare the funnel and a second amount of testing liquid for the second liquid intake (steps 4-5). After 20 minutes+/−15 seconds, repeat steps 6 through 9 for the second liquid intake.

11. Repeat step 10 for the third liquid intake.

12. Immediately after the third insult is absorbed, reset the timer for one minute. During that interval, remove the funnel and lift the top section of the top FIFE board off of the specimen. Holding the specimen as close to horizontal as possible, place the specimen on the saturated capacity tester (described below) with the liner side of the specimen facing up. Do not touch the test area. Tare out two blotters on the scale. NOTE: An alternative to taring out the weight of the blotters is to weigh the blotters dry, weigh the blotters wet, and then to subtract dry weight from the wet weight to get amount of flowback. Place the two blotter papers one on top of the other, over the target zone on the specimen. Cover the specimen and blotters with the rubber dam, taking care to make the dam taut, but not so tight that fluid is forced out of the specimen.

13. Adjust the vacuum valve to read 14+/−1 inches of water (3.4+/−0.3 kPa) on the vacuum gauge; the time needed to reach this value should be no more than 30 seconds. Hold this pressure for two minutes.

14. After two minutes lift the rubber dam to release the pressure.

15. Immediately weigh the wet blotters and record the weight.

16. The amount of liquid flowback (Flowback index) is calculated as follows: the mass difference between wet and dry blotters.

### Caliper and Density

A region of the absorbent structure 50 to be tested is placed under a 0.2 psi weight and the caliper (i.e. the thickness in the Z-direction 52) in this region is recorded. The area under compression should be at least a 2-inch by 2-inch (5.08 cm by 5.08 cm) square. A suitable tester for absorbent caliper is a STARRET-type bulk tester equipped with a 2-inch diameter brass foot that applies a weight of 0.2 psi.

The area under compression is marked around the perimeter of the weight while the weight is in place. The weight is removed and a 2-inch by 2-inch (5.08 cm by 5.08 cm) square is cut out from within the outlined region, such as by a die cut. Any tissue present on the absorbent structure 50 is removed, and the square is weighed. The density is determined by the following calculation: mass of absorbent in g/((5.08 cm)²×(bulk in cm)).

### Material Flexibility

This procedure is a single-cycle compression bench test to measure the force required to deflect a segment of a material sample into a circular orifice to a fixed distance. The procedure measures load values when the material is pressed into the orifice by a probe. The peak load value is reported.

Data generated by this test method include: Load values as the material is being deflected into the orifice.

Overview and Specimen Preparation:

A material sample is pressed into a circular orifice by a plunger on a tensile test unit; the peak load value is measured as the probe presses to a fixed distance. The term “load” refers to the gram value measured by the load cell in the tensile tester; the load cell must be capable of testing compression.

A material sample is prepared by cutting specimens from sample products or materials. A die cut may be used for this purpose. Specimen dimension is a square 37.5 mm by 37.5 mm; when taken from an intact product, the straight edges of the square should be aligned with the longitudinal and lateral axes of the product. Specimens should be obtained from areas of the product or material that have no wrinkles or folds, and have not been subjected to bending or creasing. Where specimens possess multiple layers not bonded or joined together, special care should be taken to maintain the original relative orientation of layers, and to keep edges of all component layers flush.

Suitable materials include absorbent composites that are the subject matter of this invention. For testing of absorbent materials alone, all material layers that are not integral to (i.e. bonded or adhered or otherwise commingled with) the absorbent structure in a product to be tested, for example facing layers, wrap sheets of tissue or nonwoven, or other product layers such as bodyside liner, outer cover, etc., should be removed prior to testing.

Where a sample is prepared from a manufactured web (prior to its incorporation in a product), specimens should be obtained from a segment of the web with consistent and even formation, such as along the midline of the web in the machine direction. Samples from the lateral (cross direction) edges of a web should be avoided, as these may suffer from edge effects in material preparation, and thus exhibit inconsistent results in testing.

Elastics that may be present in a product should be removed prior to obtaining specimens, taking care to avoid cutting, compressing, or distorting the absorbent core during removal. Specimens should be tested with the body-side of the absorbent (or product) facing the test probe.

Apparatus and Materials:

Tensile tester: MTS tensile tester model Synergie 200 Test Bed; available from MTS® Systems Corporation, Research Triangle Park, North Carolina USA, or an equivalent system, preferably equipped with a computerized data-acquisition system capable of calculating a peak load in grams.

Load cells: A suitable cell selected so the majority of the peak load values fall between the manufacturer's recommended ranges of load cell's full scale value; for example, a 2,000 g load cell may be appropriate, such as may be obtained from MTS® Systems Corporation, Research Triangle Park, North Carolina USA. The load cell must be able to measure compression.

Operating software and data acquisition system: MTS TestWorks® for Windows software version 4; available from MTS® Systems Corporation, Research Triangle Park, North Carolina USA, or an equivalent system for the tensile tester used.

The apparatus employed is a modified Circular Bend Stiffness Tester, having the following parts: a smooth-polished metal plate platform which is 110 mm (length) by 102 mm (width) by 6.35 mm (depth) having a 18.75 mm diameter orifice. The lap edge of the orifice should be at a 45 degree angle to a depth of 4.75 mm.

A plunger having the following dimensions is used: overall length of 100 mm, a diameter of 6.25 mm, a ball nose having a radius of 2.97 mm and a needle-point extending about 1 mm from the ball nose with a 0.33 mm base diameter and a point having a radius of less than 0.5 mm. The plunger is mounted concentrically with the orifice having equal clearance on all sides. The needle-point is used merely to prevent lateral movement of a sample during testing. The bottom of the plunger should be set well above the top of the orifice plate. From this position, the downward stroke of the ball nose is to the exact bottom of the plate orifice.

Sample Conditioning

Reasonable ambient conditions should be used for sample testing, such as 73+/−2 degrees Fahrenheit and a relative humidity of 50+/−2%. Samples should be allowed to equilibrate to laboratory conditions for at least two hours prior to testing.

Apparatus Preparation and Procedure

Tensile Tester Test Conditions:

| Data acquisition rate | 100 Hz |
|---|---|
| Crosshead speed | 500 +/− 10 mm/min |
| Full scale load: | 2,000 g |
| Gage length: | 25.4 +/− 1 mm |
| Number of cycles: | 1 |

A. Center a specimen over the orifice.

B. Start the crosshead.

C. When the test is finished and the crosshead has returned, remove the specimen from the orifice.

D. Repeat the above steps for each additional specimen.

E. Continue testing all samples in this manner.

F. Report data for each sample in the following way:

Average Peak Load

A specimen with a peak load that exceeds the limits of the load cell (˜2,000 g) should have a peak load listed as >2,000 g. The average calculation for that sample should use 2,000 g as the peak load for that specimen, with a notation made that the average is conservative (low) due to rounding down at least one peak load level to 2,000 g.

### Saturated Capacity

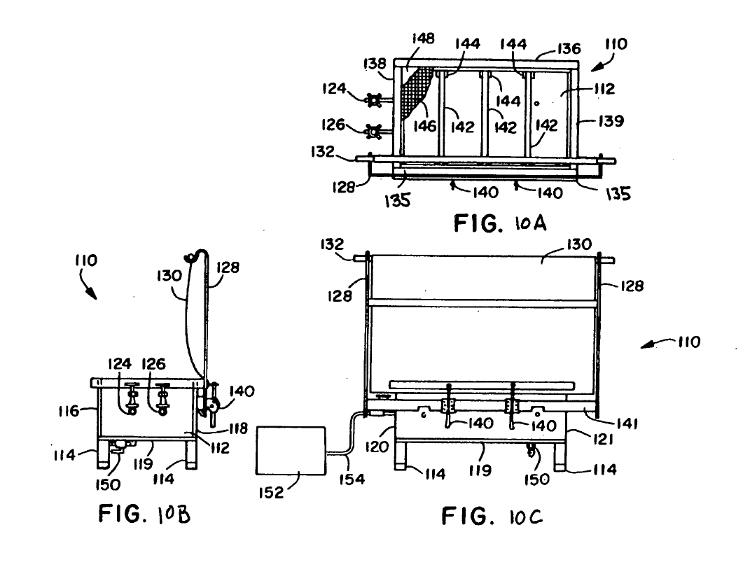

Saturated capacity is determined using a Saturated Capacity (SAT CAP) Tester with Magnahelic vacuum gage and latex dam. Referring to FIGS. 10A-10C, a Saturated Capacity tester vacuum apparatus 110 comprises a vacuum chamber 112 supported on four leg members 114. Vacuum chamber 112 includes a front wall member 116, a rear wall member 118 and two side walls 120 and 121. The wall members are about 0.5 inches thick, and are constructed and arranged to provide a chamber having outside dimensions measuring 23.5 inches in length, 14 inches in width and 8 inches in depth. A vacuum pump (not shown) operably connects with vacuum chamber 112 through an appropriate vacuum line conduit and vacuum valve 124.

In addition, a suitable air bleed line connects into vacuum chamber 112 through air bleed valve 126. A hanger assembly 128 is suitably mounted on rear wall 118 and is configured with S-curved ends to provide a convenient resting place for supporting latex dam sheet 130 in a convenient position away from the top of vacuum apparatus 110. A suitable hanger assembly can be constructed from 0.25 inch diameter stainless steel rod. Latex sheet 130 is looped around dowel member 132 to facilitate grasping and allow a convenient movement and positioning of the latex sheet. In the illustrated position, dowel member 132 is shown supported in hanger assembly 128 to position the latex sheet 130 in an open position away from the top of vacuum chamber 112. A bottom edge of latex sheet 130 is clamped against a rear edge support member 135 with suitable securing means, such as toggle clamps 140. The toggle clamps are mounted on rear wall member 118 with suitable spacers 141 which provide an appropriate orientation and alignment of the toggle clamps for the desired operation. Three support shafts 142 are 0.75 inches in diameter and are removably mounted within vacuum chamber 112 by means of support brackets 144. The support brackets are generally equally spaced along front wall member 116 and rear wall member 118 and arranged in cooperating pairs. In addition, the support brackets are constructed and arranged to suitably position the uppermost portions of support shafts 142 flush with the top of the front, rear and side wall members of vacuum chamber 112. Thus, support shafts 142 are positioned substantially parallel with one another and are generally aligned with side wall members 120 and 121.

In addition to rear edge support member 135, the tester apparatus includes a front support member 136 and two side support members 138 and 139. Each edge support member measures about 1 inch in width and about 1.25 inches in height. The lengths of the support members are constructed to suitably surround the periphery of the open top edges of vacuum chamber 112, and are positioned to protrude above the top edges of the chamber wall members by a distance of about 0.5 inches. A layer of egg crating type material 146 is positioned on top of support shafts 142 and the top edges of the wall members of vacuum chamber 112. The egg crate material extends over a generally rectangular area measuring 23.5 inches by 14 inches, and has a depth measurement of about 0.38 inches. The individual cells of the egg crating structure measure about 0.5 inch square, and the thin sheet material comprising the egg crating is composed of a suitable material, such as polystyrene. For example, the egg crating material can be McMaster Supply Catalog No. 162 4K 14, translucent diffuser panel material. A layer of 0.19 mesh nylon screening 148, which measures 23.5 inches by 14 inches, is placed on top of egg crating material 146. A suitable drain line and drain valve 150 connects to bottom plate member 119 of vacuum chamber 112 to provide a convenient mechanism for draining liquids from the vacuum chamber.

The various wall members and support members of tester 110 may be composed of a suitable non-corroding, moisture-resistant material, such as polycarbonate plastic. The various assembly joints may be affixed by solvent welding, and the finished assembly of the tester is constructed to be watertight. A vacuum gauge 152 operably connects through a conduit into vacuum chamber 112. A suitable pressure gauge is a Magnahelic differential gauge capable of measuring a vacuum of 0-100 inches of water, such as a No. 2100 gauge available from Dwyer Instrument Incorporated.

The dry product or other absorbent structure is weighed and then placed in excess 0.9% saline solution and allowed to soak for 20 minutes. After the 20 minute soak time, the absorbent structure is placed on the egg crate material and mesh nylon screening of the Saturated Capacity Tester. The latex sheet is placed over the absorbent structure(s) and the entire egg crate grid so that the sheet creates a seal when vacuum is drawn on the Tester. A vacuum of 0.5 pounds per square inch (psi) is held in the Saturated Capacity tester for five minutes. The vacuum creates a pressure on the absorbent structure(s), causing drainage of some liquid. After five minutes at 0.5 psi vacuum, the latex sheet is rolled back and the absorbent structure(s) are weighed to generate a wet weight.

The overall absorbent capacity of each absorbent structure is determined by subtracting the dry weight of each absorbent from the wet weight of that absorbent determined at this point in the procedure. The 0.5 psi SAT CAP or SAT CAP of the absorbent structure is determined by the following formula: (wet weight−dry weight)/(dry weight). This SAT CAP value has units of grams fluid/gram absorbent. For both overall capacity and SAT CAP, a minimum of four specimens of each sample should be tested, and the results averaged. If the absorbent structure has low integrity or disintegrates during the soak or transfer procedures, the absorbent structure can be wrapped in a containment material such as paper toweling, for example VIVA® paper towels manufactured by Kimberly-Clark Corporation, Neenah, Wis. The absorbent structure can be tested with the overwrap in place, and the capacity of the overwrap can be independently determined and subtracted from the wet weight of the total wrapped absorbent structure to obtain a wet absorbent weight.

### Edgewise Compression Test Method

The method by which the Edgewise Compression (EC) value can be determined is set forth below. A material sample is prepared by cutting specimens from sample products or materials. Specimen dimensions are a 2-inch by 12-inch (5.1 cm by 30.5 cm) and may include the thickest part of the product. The specimen is cut with its longer dimension aligned with the longitudinal direction of the product or raw material web. Where specimens possess multiple layers not bonded or joined together, special care should be taken to maintain the original relative orientation of layers, and to keep edges of all component layers flush.

Suitable materials include absorbent composites that are the subject matter of this invention. For testing of absorbent materials alone, all material layers that are not integral to (i.e. bonded or adhered or otherwise commingled with) the absorbent structure in a product to be tested, for example facing layers, wrap sheets of tissue or nonwoven, or other product layers such as bodyside liner, outer cover, etc., should be removed prior to testing.

Where a sample is prepared from a manufactured web (prior to its incorporation in a product), specimens should be obtained from a segment of the web with consistent and even formation, such as along the midline of the web in the machine direction. Samples from the lateral (cross direction) edges of a web should be avoided, as these may suffer from edge effects in material preparation, and thus exhibit inconsistent results in testing.

Elastics that may be present in a product should be removed prior to obtaining specimens, taking care to avoid cutting, compressing, or distorting the absorbent core during removal.

The weight of the sample is determined. The thickness of the material is determined under a 0.2 psi (1.38 KPa) load. The material is formed into a cylinder having a height of 2 inches (5.1 cm), and with the two ends having 0-0.125 inch (0-3.18 mm) overlap, the material is stapled together with three staples. One staple is near the middle of the width of the product, the other two nearer each edge of the width of the material. The longest dimension of the staple is in the circumference of the formed cylinder to minimize the effect of the staples on the testing.

An INSTRON tester, or similar instrument, is configured with a bottom platform, an upper platen larger than the circumference of the sample to be tested and parallel to the bottom platform, attached to a compression load cell placed in the inverted position. The specimen is placed on the platform, under the platen. The platen is brought into contact with the specimen and compresses the sample at a rate of 25 mm/min. The maximum force obtained in compressing the sample to 50% of its width (1 inch) (2.54 cm) is recorded.

If the material buckles, it is typical for the maximum force to be reached before the sample is compressed to 50%. In a product where the length of the absorbent is less than 12 inches (30.5 cm), the EC value of the material can be determined in the following manner. A detailed discussion of the edge-wise compression strength has been given in The Handbook Of Physical And Mechanical Testing Of Paper And Paperboard, Richard E. Mark editor, Dekker 1983 (Vol. 1). Based on theoretical models governing buckling stresses, in the Edge-wise Compression configuration described, the buckling stress is proportional to E*t²/(H²) with the proportionality constant being a function of H²/(R*t) where E is the Elastic modulus, H is the height of the cylinder, R is the radius of the cylinder, and t is the thickness of the material. Expressing the stress in terms of force per basis weight, it can be shown that the parameter that needs to be maintained constant is H²/R. Therefore, for a sample that is smaller than 12 inches (30.5 cm), the largest possible circle should be constructed and its height (width of the sample being cut out) adjusted such that H²/R equals 2.1 inches (5.3 cm).

TABLE 1
RESULTS

| | | | | Material | Material | | | | |
|---|---|---|---|---|---|---|---|---|---|
| | | | | Flexibility | Flexibility | Material | Material | | Absorbent |
| | | | | (Abs. | (Abs. | Flexibility | Flexibility | | Structure |
| | SAM | Front | Back | Structure- | Structure- | (Product- | (Product- | | Edge-Wise |
| Example | (Wt) | Density | Density | Front) | Back) | Front) | Back) | Product | Compression |
| set | % | (g/cc) | (g/cc) | (g) | (g) | (g) | (g) | Gender | (g) |
| 1 | 56% | 0.36 | 0.33 | not | not | not | not | Male | 1133 |
| | | | | tested | tested | tested | tested | | |
| | 60% | 0.34 | 0.34 | not | not | not | not | Male | 836 |
| | | | | tested | tested | tested | tested | | |
| | 65% | 0.35 | 0.40 | not | not | not | not | Male | 740 |
| | | | | tested | tested | tested | tested | | |
| | 70% | 0.33 | 0.39 | not | not | not | not | Male | 412 |
| | | | | tested | tested | tested | tested | | |
| 2 | 55% | 0.33 | 0.31 | 166 | 146 | 583 | 288 | Male | 648 |
| | 62% | 0.31 | 0.36 | 64 | 97 | 332 | 179 | Male | 399 |
| | 70% | 0.34 | 0.40 | 56 | 106 | 197 | 193 | Male | 426 |
| 3 | 55% | 0.38 | 0.35 | 298 | 259 | 1187 | 1017 | Male | 1471 |
| | 60% | 0.33 | 0.34 | 224 | 264 | 498 | 490 | Male | 692 |
| | 66% | 0.36 | 0.37 | 103 | 195 | 426 | 458 | Male | 477 |
| | 71% | 0.35 | 0.34 | 61 | 159 | 255 | 526 | Male | 227 |
| 4 | 54% | 0.36 | 0.34 | 135 | 204 | 319 | 535 | Male | not tested |
| | 66% | 0.42 | 0.44 | 86 | 101 | 208 | 345 | Male | not tested |
| | 64% | 0.37 | 0.38 | 80 | 155 | 353 | 394 | Female | not tested |
| | 52% | 0.33 | 0.33 | 302 | 201 | 473 | 469 | Female | not tested |
| | Product | | | FIFE | FIFE | FIFE | | Front | Back |
| | Edge-wise | Front | Back | Rate | Rate | Rate | | Basis | Basis |
| Example | Compression | Caliper | Caliper | Insult 1 | Insult 2 | Insult 3 | Flowback | Weight | Weight |
| set | (g) | (mm) | (mm) | (g/s) | (g/s) | (g/s) | (g) | (g/m²) | (g/m²) |
| 1 | not tested | 1.9 | 1.7 | 3.3 | 2.9 | 2.6 | 20.61 | 701 | 565 |
| | not tested | 1.9 | 1.6 | 3.3 | 2.9 | 2.6 | 22.81 | 662 | 552 |
| | not tested | 1.8 | 1.4 | 3.4 | 2.8 | 2.4 | 20.87 | 616 | 570 |
| | not tested | 1.8 | 1.4 | 3.9 | 3.0 | 2.4 | 20.26 | 576 | 551 |
| 2 | 1371 | 2.3 | 1.8 | 4.8 | 3.8 | 2.9 | 23.35 | 762 | 570 |
| | 1259 | 2.1 | 1.5 | 4.8 | 3.6 | 2.9 | 23.35 | 647 | 536 |
| | 1032 | 1.8 | 1.4 | 3.6 | 2.7 | 2.3 | 23.35 | 600 | 566 |
| 3 | 2709 | 1.9 | 1.3 | 4.3 | 4.4 | 4.0 | 21.49 | 691 | 450 |
| | 1985 | 1.9 | 1.4 | 4.4 | 4.3 | 3.9 | 21.20 | 629 | 490 |
| | 2022 | 1.8 | 1.4 | 3.3 | 3.3 | 3.4 | 23.64 | 623 | 513 |
| | 1082 | 1.7 | 1.3 | 4.0 | 3.5 | 3.1 | 21.31 | 592 | 450 |
| 4 | not tested | 1.9 | 1.4 | 3.6 | 3.8 | 3.2 | 21.99 | 679 | 451 |
| | not tested | 1.4 | 1.0 | 3.3 | 3.4 | 3.3 | 24.55 | 579 | 421 |
| | not tested | 1.5 | 1.1 | not | not | not | not | 549 | 405 |
| | | | | tested | tested | tested | tested | | |
| | not tested | 1.8 | 1.3 | not | not | not | not | 590 | 418 |
| | | | | tested | tested | tested | tested | | |

In view of the above, it will be seen that the several advantages of the invention are achieved and other advantageous results attained. In particular, the superabsorbent percentages of the examples above provided an absorbent body configuration where the higher density of the absorbent body yielded a relatively flexible absorbent and thus a more flexible, conforming product.

When introducing elements of the present invention or the preferred aspect(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above absorbent structure 50 without departing from the scope of the invention, it is intended that all matter contained in the above description and shown in the accompanying drawings shall be interpreted as illustrative and not in a limiting sense.

1. An absorbent structure comprising:
At least 60 weight % superabsorbent material and defining a density of at least 0.30 g/cm³ and a flexibility of less than 150 grams as determined by the Material Flexibility Test set forth herein.

2. The absorbent structure of claim 1 wherein said absorbent structure comprises at least 70 weight % superabsorbent.

3. The absorbent structure of claim 1 wherein said absorbent structure comprises from 60 weight % superabsorbent to 75 weight % superabsorbent.

4. The absorbent structure of claim 1 wherein said absorbent structure defines a density of at least 0.40 g/cm³.

5. The absorbent structure of claim 1 wherein said absorbent structure defines a density of at least 0.45 g/cm³.

6. The absorbent structure of claim 1 wherein said absorbent structure defines a density of from 0.33 g/cm³ to 0.43 g/cm³.

7. The absorbent structure of claim 1 wherein said absorbent structure defines an overall absorbent capacity of at least 300 g as determined by the saturated capacity (SAT CAP) test set forth herein.

8. The absorbent structure of claim 7 wherein said absorbent structure defines an overall absorbent capacity of at least 1000 g as determined by the SAT CAP test set forth herein.

9. The absorbent structure of claim 1 wherein said absorbent structure defines an edgewise compression of less than 800 g as determined by the Edgewise Compression Test set forth herein.

10. The absorbent structure of claim 9 wherein said absorbent structure defines an edgewise compression of less than 500 g as determined by the Edgewise Compression Test set forth herein.

11. The absorbent structure of claim 1 wherein said absorbent structure defines a material flexibility of less than 100 g as determined by the Material Flexibility Test set forth herein.

12. The absorbent structure of claim 1 wherein said absorbent structure defines a material flexibility of less than 50 g as determined by the Material Flexibility Test set forth herein.

13. An absorbent structure comprising:
At least 60 weight % superabsorbent material and defining a density of at least 0.30 g/cm³ and an edgewise compression of less than 800 g as determined by the Edgewise Compression Test set forth herein.

14. An absorbent structure comprising:
At least 60 weight % superabsorbent material and defining a flexibility of less than 150 grams as determined by the Material Flexibility Test set forth herein and an edgewise compression of less than 800 g as determined by the Edgewise Compression Test set forth herein.

15. A disposable absorbent article defining a first waist region and a second waist region, said article comprising:
A liquid impermeable outer cover;
A fluid permeable liner; and
An absorbent structure disposed between said outer cover and said liner, said absorbent structure comprising at least 60% superabsorbent material and defining a density of at least 0.30 g/cm³ and a level of flexibility of less than 150 g as determined by the Material Flexibility Test set forth herein; and
A fastening system configured to join said first waist region to said second waist region to define a waist opening and a pair of leg openings.

16. The absorbent article of claim 15 wherein said absorbent structure comprises at least 70 weight % superabsorbent.

17. The absorbent article of claim 15 wherein said absorbent structure comprises from 60 weight % superabsorbent to 75 weight % superabsorbent.

18. The absorbent article of claim 15 wherein said absorbent structure defines a density of at least 0.40 g/cm³.

19. The absorbent article of claim 15 wherein said absorbent structure defines a density of at least 0.45 g/cm³.

20. The absorbent article of claim 15 wherein said absorbent structure defines a density of from 0.33 g/cm³ to 0.43 g/cm³.

21. The absorbent article of claim 15 wherein said absorbent structure defines an overall absorbent capacity of at least 1000 g as determined by the saturated capacity (SAT CAP) test set forth herein.

22. The absorbent article of claim 15 wherein said absorbent structure defines an edgewise compression of less than 500 g as determined by the Edgewise Compression Test set forth herein.

23. The absorbent article of claim 15 wherein said absorbent structure defines a material flexibility of less than 50 g as determined by the Material Flexibility Test set forth herein.
